(19) United States
(12) Patent Application Publication
(10) Pub. No.: US 2008/0027008 A1
(43) Pub. Date: Jan. 31, 2008

(54) Antitumorigenic Drug Combination

(76) Inventor: Jack Henkin, Highland Park, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/831,047

(22) Filed: Jul. 31, 2007

Related U.S. Application Data
(60) Provisional application No. 60/820,873, filed on Jul. 31, 2006.

(51) Int. Cl. A61K 38/08; A61P 35/00

(52) U.S. Cl. 514/15; 514/789

(57) ABSTRACT

Compositions comprising drugs having cooperative activity and methods of treatment using the combinations are disclosed.

This application claims priority to the provisional application Ser. No. 60/820,873 filed on Jul. 31, 2006.

### FIELD OF THE INVENTION

The invention relates to compositions comprising drugs having cooperative activity and methods of treatment using the combinations.

### BACKGROUND OF THE INVENTION

Non-Hodgkin's lymphomas (NHLs) are a heterogeneous group of lymphoproliferative malignancies with differing patterns of behavior and responses to treatment. NHL usually originates in lymphoid tissues and can spread to other organs. NHL, however, is much less predictable than Hodgkin's lymphoma and has a far greater predilection to disseminate to extranodal sites. The prognosis depends on the histologic type, stage, and treatment. Every year there are over 58000 new cases of NHL and almost 20000 deaths. There is thus a great need to advance the care and treatment of the disease.

The object of this invention is a combination therapy of N-[2-[(4-HYDROXYPHENYL)AMINO]-3-PYRIDYL]-4-METHOXYBENZENESULFONAMIDE, an orally available antimitotic agent, and ABT-510, an antiangiogenic peptide with the following sequence: N—Ac—Sar-Gly-Val-D-alloIle-Thr-Nva-Ile-Arg-Pro-NHCH₂CH₃.

### SUMMARY OF THE INVENTION

Accordingly, one embodiment of this invention pertains to compositions for treating cancer in a mammal, said composition comprising therapeutically effective amounts of an antimitotic agent and a peptidomimetic of the second of the three Type-1 repeats of thrombospondin-1 (TSP-1) or a salt, prodrug or salt of a prodrug of either or both.

Still another embodiment pertains to separate compositions to be administered together for treating cancer in a mammal, one comprising a therapeutically effective amount of an antimitotic agent or a salt, prodrug, or salt of a prodrug thereof, and the other comprising a peptidomimetic of the second of the three Type-1 repeats of TSP-1 or a salt, prodrug or salt of a prodrug thereof.

Still another embodiment pertains to compositions for treating cancer in a mammal, said compositions comprising therapeutically effective amounts of an antimitotic agent and N—Ac-Sar-Gly-Val-D-alloIle-Thr-Nva-Ile-Arg-Pro-NHCH₂CH₃ or a salt, prodrug or salt of a prodrug of either or both.

Still another embodiment pertains to separate compositions to be administered together for treating cancer in a mammal, one comprising a therapeutically effective amount of an antimitotic agent or a salt, prodrug or salt of a prodrug thereof, and the other comprising N—Ac-Sar-Gly-Val-D-alloIle-Thr-Nva-Ile-Arg-Pro-NHCH₂CH₃ or a salt, prodrug or salt of a prodrug thereof.

Still another embodiment pertains to compositions for treating cancer in a mammal, said compositions comprising therapeutically effective amounts of N-[2-[(4-HYDROXYPHENYL)AMINO]-3-PYRIDYL]-4-METHOXYBENZENESULFONAMIDE and a peptidomimetic of the second of the three Type-1 repeats of TSP-1 or a salt, prodrug, or salt of a prodrug thereof.

Still another embodiment pertains to separate compositions to be administered together for treating cancer in a mammal, one comprising a therapeutically effective amount of N-[2-[(4-HYDROXYPHENYL)AMINO]-3-PYRIDYL]-4-METHOXYBENZENESULFONAMIDE and the other comprising a peptidomimetic of the second of the three Type-1 repeats of TSP-1 or a salt, prodrug, or salt of a prodrug thereof.

Still another embodiment pertains to compositions for treating cancer in a mammal, said compositions comprising therapeutically effective amounts of N-[2-[(4-HYDROXYPHENYL)AMINO]-3-PYRIDYL]-4-METHOXYBENZENESULFONAMIDE and N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to separate compositions to be administered together for treating cancer in a mammal, one comprising a therapeutically effective amount of N-[2-[(4-HYDROXYPHENYL)AMINO]-3-PYRIDYL]-4-METHOXYBENZENESULFONAMIDE and the other comprising N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to compositions for treating non-Hodgkin's lymphomas (NHLs) in a mammal, said compositions comprising therapeutically effective amounts of an antimitotic agent and a peptidomimetic of the second of the three Type-1 repeats of TSP-1 or a salt, prodrug, or salt of a prodrug of either or both.

Still another embodiment pertains to separate compositions to be administered together for treating non-Hodgkin's lymphomas (NHLs) in a mammal, one comprising a therapeutically effective amount of antimitotic agent or a salt, prodrug, or salt of a prodrug thereof, and the other comprising a peptidomimetic of the second of the three Type-1 repeats of TSP-1 or a salt, prodrug or salt of a prodrug thereof.

Still another embodiment pertains to compositions for treating non-Hodgkin's lymphomas (NHLs) in a mammal, said compositions comprising therapeutically effective amounts of antimitotic agent or a salt, prodrug, or salt of a prodrug thereof and N—Ac-Sar-Gly-Val-D-alloIle-Thr-Nva-Ile-Arg-Pro-NHCH₂CH₃, or a salt, prodrug, or salt of a prodrug of either or both.

Still another embodiment pertains to separate compositions to be administered together for treating non-Hodgkin's lymphomas (NHLs) in a mammal, one comprising a therapeutically effective amount of antimitotic agent or a salt, prodrug or salt of a prodrug thereof, and the other comprising N—Ac-Sar-Gly-Val-D-alloIle-Thr-Nva-Ile-Arg-Pro-NHCH₂CH₃ or a salt, prodrug or salt of a prodrug thereof.

Still another embodiment pertains to compositions for treating non-Hodgkin's lymphomas (NHLs) in a mammal, said compositions comprising therapeutically effective amounts of an antimitotic agent and a peptidomimetic of the second of the three Type-1 repeats of TSP-1, or a salt, prodrug, or salt of a prodrug of either or both.

Still another embodiment pertains to separate compositions to be administered together for treating lymphoma in a mammal, one comprising a therapeutically effective amount of an antimitotic agent and the other comprising a peptidomimetic of the second of the three Type-1 repeats of TSP-1 or a salt, prodrug, or salt of a prodrug thereof.

Still another embodiment pertains to compositions for treating lymphoma in a mammal, said compositions comprising therapeutically effective amounts of antimitotic agent and N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to separate compositions to be administered together for treating non-Hodgkin's lymphomas (NHLs) in a mammal, one comprising a therapeutically effective amount of N-[2-[(4-HYDROXYPHENYL)AMINO]-3-PYRIDYL]-4-METHOXYBENZENESULFONAMIDE and the other comprising N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to separate compositions to be administered together for treating non-Hodgkin's lymphomas (NHLs) in a mammal, one comprising a therapeutically effective amount of an antimitotic agent or a salt, prodrug, or salt of a prodrug thereof, and the other comprising a peptidomimetic of the second of the three Type-1 repeats TSP-1 or a salt, prodrug or salt of a prodrug thereof.

Still another embodiment pertains to separate compositions to be administered together for treating lymphomas in a mammal, one comprising a therapeutically effective amount of an antimitotic agent or a salt, prodrug or salt of a prodrug thereof, and the other comprising N—Ac-Sar-Gly-Val-D-alloIle-Thr-Nva-Ile-Arg-Pro-NHCH₂CH₃ or a salt, prodrug or salt of a prodrug thereof.

Still another embodiment pertains to methods for treating lymphoma in a mammal, said methods comprising administering to the mammal therapeutically effective amounts of N-[2-[(4-HYDROXYPHENYL)AMINO]-3-PYRIDYL]-4-METHOXYBENZENESULFONAMIDE and a peptidomimetic of the second of the three Type-1 repeats TSP-1 or a salt, prodrug or salt of a prodrug of either or both.

Still another embodiment pertains to methods for treating lymphoma in a mammal, said methods comprising administering to the mammal therapeutically effective amounts of N-[2-[(4-HYDROXYPHENYL)AMINO]-3-PYRIDYL]-4-METHOXYBENZENESULFONAMIDE and N—Ac-Sar-Gly-Val-D-alloIle-Thr-Nva-Ile-Arg-Pro-NHCH₂CH₃ or a salt, prodrug or salt of a prodrug thereof.

### DETAILED DESCRIPTION OF THE INVENTION

Variable moieties herein are represented by identifiers (capital letters with numerical and/or alphabetical superscripts) and may be specifically embodied.

Proper valences are maintained for all moieties and combinations thereof of the compounds of this invention.

The term “antimitotic agent,” as used herein, means a compound that inhibits cell growth by stopping cell division. Antimitotic agents may also be called antimicrotubule agents, mitotic inhibitors, and taxanes. Docetaxel and paclitaxel are antimitotic agents.

The term “treating,” as used herein, means at least sustaining and preferably reversing the course of a disease or adverse physiological event.

The term “cancer,” as used herein, means growth of tumor cells which interfere with the growth of healthy cells. Cancers include, but are not limited to, fibrosarcoma and gastrointestinal cancer such as gastric cancer, colon cancer and the like.

The term “mammal,” as used herein, means a particular class of vertebrate.

The term “thrombospondin-1,” as used herein, means an antiangiogenic protein which functions by inhibiting endothelial cell proliferation, thereby inducing apoptosis (programmed cell death).

The term “peptidomimetic of the second of the three Type-1 repeats of TSP-1,” as used herein, means parent peptide Gly-Val-Ile-Thr-Arg-Ile-Arg, the N-terminus Gly of which is capped with R¹-Sar, the Ile of which is replaced with D-Ile or D-alloIle, the Arg of which is replaced with Nva or Gln and the terminal Arg of which is replaced with Pro-R², wherein R¹ is hydrogen or an N-terminus prodrug-forming moiety, and R² is hydrogen or a C-terminus prodrug-forming moiety. N—Ac-Sar-Gly-Val-D-alloIle-Thr-Nva-Ile-Arg-Pro-NHCH₂CH₃ may also be referred to as ABT-510.

Compounds of this invention contain amino acids having asymmetrically substituted carbon atoms in the L- or D-configuration, wherein amino acids having the L-configuration are those which occur naturally. Atoms with an excess of one configuration over the other are assigned the configuration which is present in the higher amount, preferably an excess of about 85%-90%, more preferably an excess of about 95%-99%, and still more preferably an excess greater than about 99.5%.

The term “D-alloIle,” as used herein, means D-alloisolucyl.

The term “Arg,” as used herein, means L-argininyl.

The term “Gly,” as used herein, means L-glycyl.

The term “Gln,” as used herein, means L-glutamine.

The term “Ile,” as used herein, means L-isolucyl.

The term “Nva,” as used herein, means L-norvalinyl.

The term “Pro,” as used herein, means L-prolinyl.

The term “Sar,” as used herein, means L-sarcosyl (N-methyl-L-glycyl).

The term “Thr,” as used herein, means L-threoninyl.

The term “Val,” as used herein, means L-valyl.

The term “drugs of this invention,” as used herein, means antimitotic agents and peptidomimetics of the second of the three Type-1 repeats of TSP-1.

The term “prodrugs of this invention,” as used herein, means antimitotic agent and peptidomimetics of the second of the three Type-1 repeats of TSP-1 having attached thereto at least one prodrug-forming moiety.

Drugs of this invention may exist as an acid addition salts, basic addition salts or zwitterions. Acid addition salts are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate salts of the drugs of this invention, and prodrugs thereof, are contemplated as being embraced by this invention. Basic addition salts of the drugs of this invention are those derived from the reaction of the same with the hydroxide, carbonate or bicarbonate of cations such as lithium, sodium, potassium, calcium and magnesium.

Drugs of this invention may be administered, for example, parenterally (intramuscularly, intraperintoneally intrasternally, intravenously subcutaneously) or transdermally.

Therapeutically effective amounts of drugs of this invention depend on the recipient of treatment, the cancer being treated and severity thereof, compositions containing them, time of administration, route of administration, duration of treatment, their potency, their rate of clearance and whether or not other drugs are co-administered. The amount of a compound of a drug of this invention used to make a composition to be administered daily to a patient in a single dose or in divided doses is from about 0.05 to about 300 mg/kg (mpk) body weight. Single dose compositions contain these amounts or a combination of submultiples thereof.

Drugs of this invention may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising drugs of this invention to be administered parenterally or transdermally include, for example, 1,3-butanediol, castor oil, corn oil, cottonseed oil, dextrose, 5% glucose in water (D5W), germ oil, groundnut oil, isotonic sodium chloride solution (0.9% sodium chloride in water), liposomes, oleic acid, olive oil, peanut oil, Ringer's solution, safflower oil, sesame oil, soybean oil, U.S.P. or, water, mixtures thereof and the like.

Drugs of this invention containing NH, C(O)OH, OH or SH moieties may have attached thereto prodrug-forming moieties which are removed by metabolic processes and release the compounds having the freed NH, C(O)OH, OH or SH in vivo. Prodrugs of this invention may have modified or improved properties such as solubility and/or hydrophobicity, absorption in the gastrointestinal tract, bioavailability, reduction of site-of-administration irritation, tissue penetration, rate of clearance and the like.

In a preferred embodiment for the practice of this invention, the N-terminus (sarcosinyl) and the C-terminus (prolyl) of the representative peptidomimetic of the second of the three Type-1 repeats of TSP-of this invention have attached thereto an acetyl (CH₃C(O) or Ac) and an ethylamino moiety, respectively.

Other N-terminus prodrug forming groups include, but are not limited to, acetoxy (CH₃CO(O)), benzoyl (C₆H₅C(O)), benzoyloxy (C₆H₅CO(O)) and the like. Other C-terminus prodrug forming groups include, but are not limited to, ethyl, diethylamino and the like.

The following biological experimental is presented to provide what is believed to be the most useful and readily understood description of procedures and conceptual aspects of this invention.

### Study Design

Twenty-five (25) dogs of varying sex, weight, breed and age (5.5-14.0 yrs) were entered for the study. Table 1 demonstrates a profile summary of the dogs enrolled to this study. All dogs entered for study had received prior chemotherapy and enrolled to the study following disease relapse.

TABLE 1
Clinical description of dogs enrolled in the study.
Efficacy Study for Treatment of Dogs with Lymphoma

| Study Number | Name | Sex | Age | Breed | Stage |
|---|---|---|---|---|---|
| 001 | Aldeanna R. | FS | 10 Y | Golden Retriever | IIia |
| 002 | Ebo F. | FS | 9 y, 3 m | Mix | IIIb |
| 003 | Cali A. | FS | 7 y, 6 m | Boxer | IIa |
| 004 | Olie L. | FS | 1l y, 9 m | Beagle | IIa |
| 005 | Sable E. | FS | 5 y, 9 m | Mix | IIia |
| 006 | Taz B. | MC | 6 y, 6 m | Doberman Pinscher | IIib |
| 007 | Maddie M. | FS | 7 y | Labrador Retriever | Vb |
| 008 | Disney F. | FS | 6 y, 9 m | Border Collie | Va |
| 009 | Spencer M. | MC | 5 y, 6 m | Husky | IIib |
| 010 | Foggy P. | MC | 11 y | Belgian Shepherd | IIia |
| 011 | Casey C. | FS | 6 y, 10 m | Labrador Retriever | IIia |
| 012 | Jack T. | MC | 6 y | Harrier | IIia |
| 013 | Sheeba P. | F | 10 y | American Staffordshire Terrier | IVb |
| 014 | Ivy B. | FS | 11 y, 1 m | German Shepherd | IIIb |
| 015 | Chelsea G. | FS | 5 y, 6 m | Labrador Retriever | IVb |
| 016 | Nestle A. | FS | 14 y | Terrier Mix | Va |
| 017 | Prince W. | MC | 10 y | Cocker Spaniel | IIia |
| 018 | Doc Holiday R. | MC | 8 y, 10 m | Golden Retriever | IIia |
| 019 | Widget H. | MC | 8 y, 11 m | Bichon Frise | IIia |
| 020 | Madison L. | FS | 9 y, 7 m | Viszla | IIia |
| 021 | Tucker M. | MC | 10 y, 1 m | Beagle Mix | Iva |
| 022 | Toby L. | M | 7 y, 10 m | English Springer Spaniel | IIia |
| 023 | G. G. Smith R. | M | 7 y, 5 m | Cocker Spaniel | IVb |
| 024 | Duncan M. | MC | 10 y, 5 m | Golden Retriever | IIia |
| 025 | Sammy D. | MC | 10 y, 3 m | Border Collie | IIia |

All dogs were treated with N-[2-[(4-HYDROXYPHENYL)AMINO]-3-PYRIDYL]-4-METHOXYBENZENESULFONAMIDE at an initial dose of 350 mg/m2 PO QD for 7 days then treated at 350 mg/m2 EOD×21 days. If no significant objective response or doselimiting toxicities were noted after 28 days on therapy, a QD regimen was restarted for 7 days. Otherwise, dogs continued on EOD dosing until study discontinuation.

All dogs were treated with ABT-510 at a dose of 1 mg/kg SQ divided BID starting 24 hours after the first dose of N-[2-[(4-HYDROXYPHENYL)AMINO]-3-PYRIDYL]-4-METHOXYBENZENESULFONAMIDE was administered.

### Response Data:

Dogs treated with N-[2-[(4-HYDROXYPHENYL)AMINO]-3-PYRIDYL]-4-METHOXYBENZENESULFONAMIDE and ABT-510 therapy were evaluated at Day 0, Day 7, Day 28 then every 28 days thereafter. Assessment of tumor burden was made based on measurement of target lymph nodes (no more than 3 nodes/animal). The lymph node volume (I×w×h) was calculated for each target lymph node and assessed as a sum of total tumor burden. Standard response endpoints were evaluated based on the criteria below:
Complete response (CR) is defined as disappearance of all clinical evidence of cancer and of any signs related to the cancer.
Partial response (PR) is defined as a 50% or greater decrease in the sum of the products of measurements for representative lesions, without an increase in size of any lesions or appearance of any new lesions.
Minimal response (MR) is defined as a 25% or greater decrease in the sum of the products of measurements for representative lesions, without an increase in size of any lesions or appearance of any new lesions.
Stable disease is defined as no response or a response of less than that defined for partial response or progressive disease without appearance of any new lesions or worsening of clinical signs.
Progressive disease is defined as an unequivocal increase of at least 50% in the size of any measurable lesion or appearance of new lesions.

The Intent-to-Treat population included all 25 dogs enrolled in the study. Five (5) dogs withdrew from the study early due to their inability to receive N-[2-[(4-HYDROXYPHENYL)AMINO]-3-PYRIDYL]-4-METHOXYBENZENESULFONAMIDE and ABT-510 longer than 7 days. Four (4) of these dogs withdrew due to rapid lymphoma progression. One (1) dog withdrew due to owner decision related to side effects while receiving study agents.

The Treatment Received population included dogs who received N-[2-[(4-HYDROXYPHENYL)AMINO]-3-PYRIDYL]-4-METHOXYBENZENESULFONAMIDE and ABT-510 therapy for longer than 7 days. Of the 25 dogs enrolled in the study, 20 are included in the Treatment Received Population.

Of the dogs in the Treatment Received Population, 8 of 20 dogs had clinical responses (CR, PR, MR). Three (3) dogs in the responding population had partial responses and 5 had minimal responses. Two (2) dogs in the responding population were censored for response duration given that they exited the study due to side effects.

Five (5) dogs in the Treatment Received Population were censored for time to progression due to study exit resulting from events other than progressive disease. Four (4) of these dogs exited the study due to toxicity and 1 dog exited at owner discretion to pursue alternative therapy.

Comparison of response and time to progression data for N-[2-[(4-HYDROXYPHENYL)AMINO]-3-PYRIDYL]-4-METHOXYBENZENESULFONAMIDE plus ABT-510 versus N-[2-[(4-HYDROXYPHENYL)AMINO]-3-PYRIDYL]-4-METHOXYBENZENESULFONAMIDE alone is shown in Table 2.

TABLE 2
Clinical response, response duration, and time to progression comparison
for treatment received populations of dogs

| | Clinical Response | Response | Time to |
|---|---|---|---|
| | (Includes PR, MR) | Duration | Progression |
| Combination | 8/20 (40.0%) | Mean = 21 days | Mean = 27 days |
| Treatment | | Median = 20 days | Median = 27 days |
| Received | | | |
| Single Agent | 3/15 (20.0%) | Mean = 65 days | Mean = 21 days |
| Treatment | | Median = 24 days | Median = 18 days |
| Received | | | |

N-[2-[(4-HYDROXYPHENYL)AMINO]-3-PYRIDYL]-4-METHOXYBENZENESULFONAMIDE is clearly not a strong candidate for monotherapy in canine lymphoma. However, the use of this agent with other cytotoxic chemotherapy or as a maintenance treatment following induction chemotherapy may be a possible care path.

The foregoing is meant to illustrate the invention and not limit it to the disclosed embodiments. Variations and changes obvious to one skilled in the art are intended to be within the scope and nature of the invention as defined in the claims.

We claim:

1. A composition for treating cancer in a mammal said composition comprising therapeutically effective amounts of an antimitotic agent and a peptidomimetic of the second of the three Type-1 repeats of thrombospondin-1 (TSP-1) or a salt, prodrug or salt of a prodrug of either or both.

2. A composition for treating cancer in a mammal said composition comprising therapeutically effective amounts of N-[2-[(4-HYDROXYPHENYL)AMINO]-3-PYRIDYL]-4-METHOXYBENZENESULFONAMIDE and acetyl-sarcosine-glycine-valine-Dalloisoleucine-threonine-norvaline-isoleucine-arginine-proline-ethylamide or a salt, prodrug or salt of a prodrug of either or both.

3. The composition of claim 2 wherein the cancer is non-Hodgkin's lymphoma.

4. The co-administration of N-[2-[(4-HYDROXYPHENYL)AMINO]-3-PYRIDYL]-4-METHOXYBENZENESULFONAMIDE and acetyl-sarcosine-glycine-valine-D-alloisoleucine-threonine-norvaline-isoleucine-arginine-proline-ethylamide or a salt, prodrug or salt of a prodrug of either or both.

5. The co-administration of claim 4 wherein the of N-[2-[(4-HYDROXYPHENYL)AMINO]-3-PYRIDYL]-4-METHOXYBENZENESULFONAMIDE and acetyl-sarcosine-glycine-valine-Dalloisoleucine-threonine-norvaline-isoleucine-arginine-proline-ethylamide are given in therapeutically effective amounts to treat cancer.

6. A method for treating cancer in a mammal said method comprising administering to the mammal therapeutically effective amounts of an antimitotic agent and a peptidomimetic of the second of the three Type-1 repeats of TSP-1 or a salt, prodrug, or salt of a prodrug thereof.

7. A method for treating non-Hodgkin's lymphomas in a mammal said method comprising administering to the mammal therapeutically effective amounts of N-[2-[(4-HYDROXYPHENYL)AMINO]-3-PYRIDYL]-4-METHOXYBENZENESULFONAMIDE and acetyl-sarcosine-glycine-valine-Dalloisoleucine-threonine-norvaline-isoleucine-arginine-proline-ethylamide or a salt, prodrug or salt of a prodrug thereof.
